(19) United States
(12) Patent Application Publication
(10) Pub. No.: US 2016/0188827 A1
(43) Pub. Date: Jun. 30, 2016

(54) Hybrid Signal Acquisition And System For Combined Electroencephalography And Cardiac Electrophysiology Studies

(71) Applicant: General Electric Company, Schenectady, NY (US)

(72) Inventors: Adrian F. Warner, Wauwatosa, WI (US); Daniel Richard Schneidewend, Wauwatosa, WI (US); Rodger F. Schmit, Wauwatosa, WI (US); Nicholas P. Nekich, Wauwatosa, WI (US)

(21) Appl. No.: 14/586,299

(22) Filed: Dec. 30, 2014

(51) Int. Cl. G06F 19/00; A61B 5/0205; A61B 5/00; A61B 5/0478; A61B 5/04; A61B 5/0432; H04L 7/00; A61B 5/0408

(52) U.S. Cl. CPC G06F 19/3406; H04L 7/0016; A61B 5/0205; A61B 5/0408; A61B 5/0478; A61B 5/04012; A61B 5/0432; A61B 5/4836

(57) ABSTRACT

In the present invention, a physiological data acquisition system for obtaining information on multiple physiological parameters from sensors operably connected to the system includes an amplifier configured to receive and output signals representative of the multiple physiological parameters in a temporally synchronized format and a central processing unit (CPU) operably connected to the hybrid amplifier to monitor the signals from the amplifier. The CPU is configured to provide a display of the synchronized signals for analysis of neurogenic conditions and to activate a recording of signals received for one physiological parameter based on a triggering event determined from signals received for another physiological parameter.

### BACKGROUND OF THE INVENTION

The invention relates generally to a monitoring and/or diagnostic system for recording physiological signals generated by a patient, and more particularly to systems and methods for recording multiple physiological signals from the patient.

In monitoring or diagnostic devices that are currently utilized, the data obtained by the devices is related to a single physiological parameter or signal generated by the patient, such as an electrocardiogram (ECG) signal or an electroencephalogram (EEG) signal. While systems of this type are effective in enabling the monitoring, recording and evaluation of the specific signal received by the devices, the information received an recorded by these devices is isolated on the de ice and not readily comparable to or utilized with signals obtained via other devices or systems in order to assess any correlations between signals of these various types and determine any potential treatments as a result of those correlations.

For example, these correlations would assist in the detection and treatment of neurogenic heart disease and neuro-cardiogenic syncope which can be considered to include postural orthostatic tachycardia, syndrome (POTS), dysautonomia, and vasovagal syncope. Neuro-cardiogenic syncope is most commonly discovered in adolescents and in older adults. It is essentially a failure of the brain and the cardiovascular system (blood vessels) to adequately communicate and respond to each other. However, due to the difficulty in correlating neurological effects on the cardiac conduction system of the affected person, treatment of this condition is complicated at best.

In the prior art some systems have been developed that incorporate components for measuring or obtaining multiple physiological signals from a patient. One example of such a system is disclosed in U.S. Pat. No. 7,881,778, which is expressly incorporated by reference herein in its entirety. In the system of the '778 patent, a conventional ECG measurement unit is capable of being modularly expanded from a 5 lead ECG unit through the use of various extension units or modules to provide the system with a 12 lead ECG unit and/or an EEG signal acquisition unit. In this manner, the system can be configured to monitor both ECG and EEG signals from a patient.

However, with this system, though the ECG and EEG signals are obtained by a single system, the data relating to each of the recorded signals is obtained utilizing a separate recording unit, such that the signal data is maintained separately from one another, such that no synergistic evaluation of the recorded data can be achieved.

In another prior art monitoring system, as disclosed in U.S. Pat. No. 8,041,418, incorporated herein by reference, the system is utilized for the monitoring and treatment of neurological disorders and includes a brain monitoring element, a cardiac monitoring element, a therapy module connected to the brain monitoring element and a processor. The processor is configured to activate the therapy module upon detection of a cardiac event in the cardiac signal. In conjunction with the activation of the therapy module, the processor is further configured to monitor the brain signal and communicate to the therapy module to change the therapeutic output to the brain based upon the brain monitoring data obtained.

However, this system obtains the signals from the cardiac and neurological leads through separate and discrete amplifiers in the components of the system. As such, the signals, while obtained in the same device, are maintained separate from one another in the discrete amplifiers, which requires additional processing of the signals in order to enable the signals to be correlated with one another for use in studies of the data represented by the signals.

Therefore, it is desirable to provide a system and method of recording or capturing and displaying patient physiological signals of dissimilar type, e.g., electroencephalographic signals from a patient in conjunction with cardiac electrophysiology signals, within one complete system and against one common time constant, such that the signals are synchronized. In addition, it is desirable to have a simplified system and associated method for observing and recording brain function signals in conjunction with cardiac function signals in order to provide support to comprehensive studies of the cardiac-neurological conduction system with respect to the heart, and the abnormalities derived from said conduction system. It is also desirable it the simplified system has generic recording resources that can be allocated to EEC or ECG on the fly as required by the progression of a case in progress.

### BRIEF DESCRIPTION OF THE INVENTION

In one exemplary aspect of the invention, a monitoring and/or diagnostic system is provided that enables hybrid neurological-cardiac studies using the system as a result of the system being operable to record all necessary cardiac and neurological signals in a synchronized manner using a single signal acquisition device or hybrid amplifier. This in turn, allows the system to display the recorded and synchronized signals in a manner selected by the user of the system for examination and analysis of the neurological effect on the cardiac conduction system of the patient, and vice versa. The system provides the ability to support comprehensive study of the cardiac-neurological conduction system with respect to the heart, and the abnormalities derived from said conduction system. The system further provides the ability to allocate recording resources dynamically to either ECG or EEG functionality within the system when in operation.

According to another exemplary aspect of the invention, all physiological parameters are acquired by the system with one acquisition device and all physiological signals are acquired by the system within the same time domain, i.e., are synchronized, where the signals can be concurrently recorded in real-time, and reviewed at any point during the acquisition of the signals, or after for review, or follow-up treatment.

According to another exemplary aspect of the invention, the functionality of the system in acquiring all signals in a synchronized manner allows a user to employ the system for use in various clinical and research modalities. In this manner the system can be utilized to capture the effect of drugs administered to a patient to determine their impact on neurological function relative to the cardiac vascular system, by means of conduction abnormalities that can be synthetically induced in the lab and captured using the system. In addition, traumatic head injuries may also cause cardiac conduction abnormalities that can impact the patient, which can be monitored and recorded by the system of the invention for analysis. The system also provides capability to support research into cardioneurogenic disease and for conventional cardiac electrophysiology studies to monitor the conscious sedation state of the patient in addition to neurological-cardiac studies. The system further provides capability to diagnose stroke or other brain related vascular trauma induced as the results of cardiac intervention procedures in progress.

According to still another exemplary aspect of the invention, the system can display the signals in any order relative to any other signal, which allows a user to employ the system to follow any individual, signal from the source of the signal to the receptor to the resulting cardiac function.

According to still a further exemplary aspect of the invention, the system can be configured for triggering from any signal source to the system. For example, a potential in the brain sensed by the system can be used to “trigger-on” a cardiac event and capture all associated cardiac signals being acquired including but not limited to iECG, ECG, BP, NIBP, and all neurological activity occurring over the same time period. The system can also capture signals from the bundle of HIS and/or anywhere within the cardiac tissue that are used to trigger-on the system to synchronously record the cardiac signals and associated neurological signals.

According to still a further exemplary aspect of the invention, the synchronous recording of the signals relating to the cardiac cycle is captured by the system in a manner that is easily removed from the EEG (i.e. non-cardiac) brain signal for analysis.

According to still another exemplary aspect of the invention, the system and method can be employed to examine and diagnose patients with cardioneurogenic, neurogenic disorders, as well as to research neurogenic disorders and diseases, to observe and detect on-set of stroke during medical procedures and to support conscious sedation monitoring within the system formed of an electrophysiological recorder.

According to still a further exemplary aspect of the invention, the invention is a physiological data acquisition system for obtaining information from sensors operably connected to the system including an amplifier, a first set of leads operably connected between a first set of sensors and the amplifier for transmitting a first set of physiological signals from the first set of sensors to the amplifier, a second set of leads operably connected between a second set of sensors and the amplifier for transmitting a second set of physiological signals from the second set of sensors to the amplifier and a CPU operably connected to the amplifier to monitor the first set of signals and the second set of signals output from the amplifier.

According to still another exemplary aspect of the invention, the invention is a method for diagnosing and treating a cardioneurogenic condition including the steps of providing a physiological data acquisition system including an amplifier, a first set of leads operably connected between a first set of sensors and the amplifier for transmitting a first set of physiological signals from the first set of sensors to the amplifier, a second set of leads operably connected between a second set of sensors and the amplifier for transmitting a second set of physiological signals from the second set of sensors to the amplifier and a CPU operably connected to the amplifier to monitor the first set of signals and the second set of signals output from the amplifier, synchronizing the first set of signals and the second set of signals in the amplifier with respect each other and recording at least one of the synchronized first set of signals and second set of signals for review and diagnosis of the cardioneurogenic condition.

According to still a further aspect of the invention, a physiological data acquisition system for obtaining information on multiple physiological parameters from sensors operably connected to the system includes an amplifier configured to receive and output signals representative of the multiple physiological parameters in a temporally synchronized format and a CPU operably connected to the amplifier to monitor the signals from the amplifier.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The drawings illustrate the best mode presently contemplated of carrying out the disclosure. In the drawings:

FIG. 1 is a schematic view of a monitoring device in accordance with an exemplary embodiment of the invention.

FIG. 2 is a schematic view of a display showing the individual and combined outputs of the device of FIG. 1

FIG. 3 is a schematic view of the method of operation of the system of FIG. 1 in accordance with an exemplary embodiment of the invention.

### DETAILED DESCRIPTION OF THE INVENTION

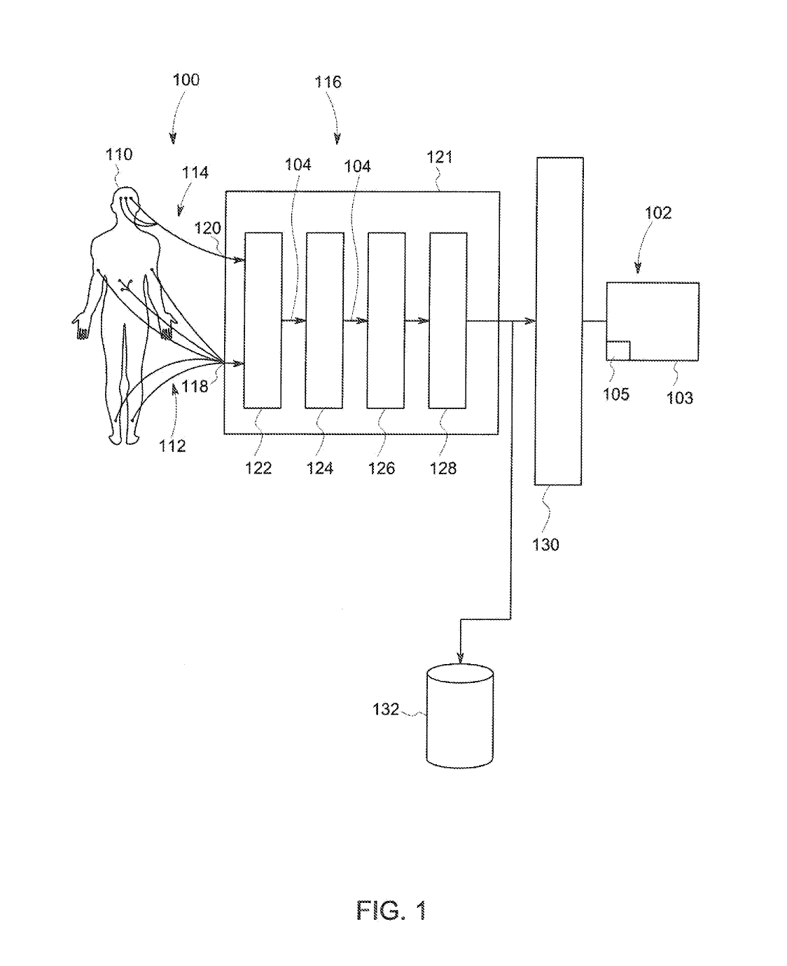

FIG. 1 illustrates an exemplary schematic embodiment of the invention which includes a monitoring or diagnostic system 100, which can be any suitable type of monitoring device for monitoring various operating parameters of a patient 110. The system 100 includes a display 102 of any suitable type, such as a touch screen display, having a screen 103 thereon on which the data signals 104 obtained from the patient 110 connected to the system 100 can be displayed. When formed as a touch screen, the display 102 can additionally function as a user interface 105 for use in controlling the operation of the system 100, though the interface 105 can be formed as a separate component connected to the system 100, if desired.

The system 100 includes a number of sensor leads 112 and 114 that are connected to a central component or device 116 at one end, and to the patient 110 at the opposite end. The leads 112 and 114 are configured and positioned on the patient 110 in order to monitor cardiac and neurological signals, respectively, from the patient 110. The leads 112 for the cardiac signal monitoring can include/employ any suitable type of cardiac sensors thereon, such as surface ECG or intracardiac sensor devices (not shown), among others known in the relevant art. Further, the leads 114 for the neurological signal monitoring can include/employ and suitable type of neurological sensors thereon, such as surface EEG or intracranial sensor devices, among others known in the relevant art. In an exemplary embodiment, the system 100 captures of intracardiac signals 104 via the leads 112, along with signals 104 associated with EEG, i.e. scalp surface based signals from the brain via leads 114. Other signals can also be acquired in conjunction with or a substitutes for these signals 104, such as surface ECG, but all signals 104 from the leads 112, 114 are acquired by the system 100 within the one signal acquisition device 116.

These leads 112 and 114 are connected to the central signal acquisition device 116 such that the signals 104 from the patient 110 are transmitted from the sensors along the respective leads 112 or 114 to the device 116. The device 116 is any suitable signal recording device, such as a cardiac EP or electrocardiogram recorder, or an EEG recorder that is capable of receiving the signals from the leads 112 and 114. Examples of the types of devices that can function/operate as the device 116 include, but are not limited to those disclosed in U.S. Pat. Nos. 7,881,778 and 8,041,418, which are incorporated by reference herein in their entirety.

As illustrated in FIG. 1, the signal acquisition device 116 includes inputs 118 and 120 for the leads 112 and 114, respectively, that are operably connected to an amplifier 121 within the device 116. The circuitry for the amplifier 121 can have any suitable configuration in a typical recording device 116, so long as the circuitry for the amplifier 121 is capable of receiving the signals 104 form each of the leads 112 and 114 for simultaneous processing and/or recording. In the illustrated exemplary embodiment of FIG. 1, the device 116 includes only a single hybrid amplifier 121, and the inputs 118, 120 are each operably connected within the amplifier 121 to a patient isolation circuit 122 provided to electrically isolate the patient 110 from electric shock hazards. The signals 104 are output from the isolation circuit 122 to a number of signal filters 124, such as low and high pass notch signal filters, in order to condition the signals 104 from the leads 112, 114 to reduce any noise within the signals 104. In addition, the signal filters 124 can be utilized to remove aspects of the various signals 104 from one another to prevent interference with one another, such as by removing the cardiac cycle from the EEG waveforms represented in the data signals 104 from the leads 114.

From the filters 124, the signals 104 are output to an analog-to-digital converter 126 capable of rendering the signals 104 from their analog form into digital form 104a for further processing by the device 116. The digital signals 104 from the converter 126 are subsequently output to a direct memory access (DMA) engine 128 that functions to transfer the digitized data signals 104 between a central processing unit (CPU) 130 and a storage database 132 for the signals 104 to enable the use of the signals 104 in various analyses performed by the CPU 130 under the direction of the user of the system 100.

The data provided by the signals 104 is output from the amplifier 121 of the acquisition device 116 to the CPU 130 where the data signals 104 are utilized in the various analyses performed by the device 116. Further, the signals 104 are synchronized at the device 116, such as within the amplifier 121 and/or the CPU 130, such that the signals 104 associated with the cardiac leads 112 and the neurological leads 114 can be correlated with time as a constant, and reviewed in direct comparison with one another utilizing the system 100. As all of the signals or channels 104 are acquired in time synchronicity by the amplifier 121, the signals 104 from the leads 112 and 114 have a time and spatial connection relative to the patient 110 that is utilized for subsequent display and analysis of the data contained within the signals 104.

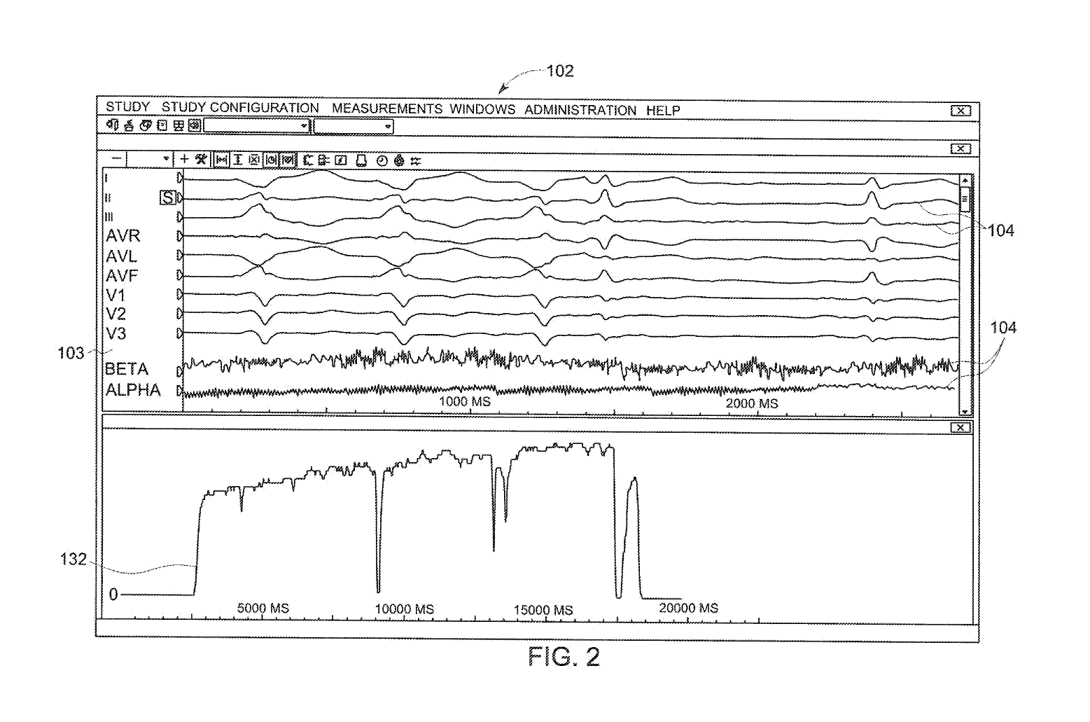

In one exemplary embodiment, referring to FIG. 2, the CPU 130 can output the signals 104 from the system 100 on the display 103 in a form that represents the signals 104 in direct comparison with one another, such as with the illustrated signals 104 displayed in direct temporal synchronicity or relation to one another. The system 100 thus has the ability to show any combination of the signals 104 in a time coordinated fashion on the display 103. In the exemplary embodiment illustrated in FIG. 2, the system 100 can display a series of neurological waveforms from the signals 104 on leads 114 with ECG waveforms obtained from signals 104 obtained via leads 112. In this manner, the system 100 provides the user with the capability to review the data signals 104 directly with one another to observe effects of various conditions, stimuli, etc. on the signals 104 received by the leads 112 and 114. In still another exemplary embodiment, the data signals 104 from the leads 112, 114 can be illustrated on the display 103 in real-time as the signals 104 are received from the leads 112 and 114, allowing for more effective monitoring of the patient 110 connected to the device 116.

The display 103 can also illustrate information about the patient 110 that is derived from the analysis of the data signals 104. In an exemplary embodiment, the display 103 can illustrate a patient status index value 132. The index value 132 is determined by the CPU 130 utilizing the EEG data signals 104 to determine a status parameter for the patient 110 based on the information about the patient 110 contained within the data signals 104. This value 132 is graphically illustrated on the display 103 over time to provide the user with a single viewable parameter indicative of the condition of the cardio-neurological conduction system for the patient 110. The index value 132 thus provides the user with a single reviewable real-time indicator/indictation of the condition of the conduction system for the patient 110 which is correlated or synchronized in time with the actual data signals 104 from which the index value 132 was derived. Therefore, should the index value 132 indicate a condition or event occurring in the patient 110, the user can easily obtain and review the synchronized data signals 104 to determine the cause(s) of the event, and the necessary treatment. In one exemplary embodiment, the index value 132 can be calculated from the weighted averages of the EEG amplitudes of the primary frequencies of interest in Alpha and Beta waves.

In addition to providing the synchronized data signals 104 concerning the dissimilar physiological parameters of the patient 110, such as in the afore-mentioned real-time display format on the display 103, the system 100 can additionally be configured in an exemplary embodiment for use in a method to trigger on or activate the recording of the data signals 104 from one or the other of the leads 112 or 114 in response to a triggering event sensed via the signals 104 being received and monitored by the device 116 and CPU 130. The ability for the system 100 to trigger or switch on the active monitoring/recording of one set of waveforms/signals 104 relative data received from the other monitored signals 104 also allows the user of the system 100 to explore potential causes and effects relative to each type of signal 104 being monitored and recorded by the system 100. In this embodiment, the device 116 actively monitors and records the data signals 104 obtained from either the cardiac leads 112 or the neurological leads 114. When an event of a specified type is determined by the system 100 using triggering event data stored in the system 100, such as in database 132, the system 100, via the CPU 130, can trigger on the monitoring of the data signals 104 from both the leads 112 and 114 to record and synchronize the data signals 104 from the leads 112 and 114 for review and analysis. The triggering, in one embodiment, can be accomplished by the amplifier 121 dynamically assigning an input or input channel of the amplifier 121 to the EEG or ECG signals 104 to direct the signals 104 from the leads 112 or 114 to the CPU 130 for recording and analysis, particularly during a procedure being monitored by the system 100. In this manner, the system 100 provides the ability to monitor the effects of procedures performed on one side of the cardiac-neurological conduction system on the opposite side of the conduction system. Specifically, in one exemplary embodiment, with inclusion of suitable pre-existing algorithm during a cardiac procedure in which the data signals 104 from the cardiac leads 112 are actively monitored, the system 100 can be employed for procedure anomaly detection. In doing so, the system 100 utilizes the EEG data signals 104 from leads 114 obtained by the system 100 during a cardiac procedure to capture and display potential neurological events that occur during the cardiac procedure, such as, for example, an ablation. The use of the system 100 can additionally be combined with other monitoring devices and/or techniques, such as pulse oximetry monitors, to capture loss of peripheral vascular circulation, a rare negative side effect of cardiac ablation where blood clots can be created and accidentally released into the vascular system where they can negatively affect neurological function, i.e., cause a stroke in the patient.

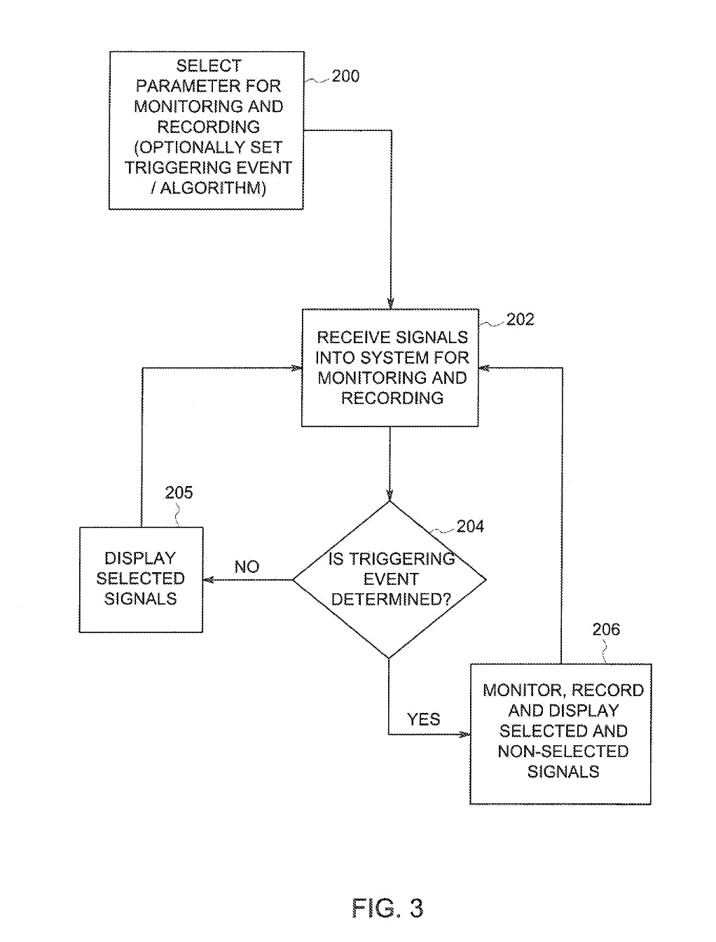

In FIG. 3, one exemplary embodiment of this triggering process is schematically illustrated. In block 200, the user selects the physiological parameter signals 104 from leads 112 or 114 that are to be monitored and recorded during the use of the system 100 during the procedure, along with optionally setting the triggering event or algorithm. One selected, the system 100 is active and begins to receive the physiological signals 104 from leads 112 and 114 in block 202, and simultaneously monitors and records the selected signals 104. In block 204, if no triggering event is determined from the selected signals 104 being monitored and recorded, the system 100 moves to block 205 and continues to receive, record and monitor the selected signals 104, but does not record the non-selected signals, and displays the selected signals 104. However, when the system 100, via CPU 130, determines that a triggering event has occurred based on the selected signal 104, the system 100 proceeds to block 206 where the signals 104 from leads 112 and 114 are both recorded. The system 100 moves back to block 202 to continue to receive the signals 104 from both sets of leads 112 and 114 until the system 100 determines the triggering event is no longer present in the originally monitored or selected signal 104 in block 204, at which time the system 100 ceases the recording of the non-selected signal 104 until a subsequent triggering event is determined, or when the procedure utilizing the system 100 is completed.

In another exemplary embodiment, the system 100 can include a suitable algorithm to be employed by the CPU 130 to the data signals 104 from the cardiac leads 112 and neurological leads 114 to actively and continuously monitor the consciousness/sedated state of a patient 110 connected to the system 100.

In still another exemplary embodiment, the system 100 can be configured to and/or utilized in the diagnosis and treatment of cardioneurogenic disease due the ability to obtain an temporally synchronize/correlate the data signals 104 from cardiac leads 112 and neurological leads 114 to one another. The system 100 can additionally capture the effects of drugs that impact neurological function by means of conduction abnormalities sensed by the system 100, and can be utilized to treat traumatic head injuries that may also cause cardiac conduction abnormalities capable of being sensed by the system 100 that can impact the patient 110.

In still another exemplary embodiment of the system 100, the system 100 can be configured to support different study types as differentiated from a classical EEG system, or standard Cardiac EP recorder. A typical configuration for the system 100 might be 64 channels (128 bi-polar) of Cardiac EP, with 20 channels (40 bi-polar) EEG signals. Other configurations could be up to 256 Cardiac EP channels and 256 EEG channels. The available configurations for the system 100 allow the system 100 to be configured in a manner unique to each type of study so that the system 100 can support multiple study configurations based on the type of procedure and/or the preference of the user. The study-based configurations available with the system 100 facilitates the ability for the user to configure the desired signals/properties illustrated on the display 103 of the system 100 such as, but not limited to, signal display order, color, gain, filters, etc. on a channel by channel basis for, e.g., ECG, BP, IECG and EEG signal types. In an alternative embodiment of the system 100, the device 116/CPU 130 is/are configured to be able to Thus, the user can operate the system 100 to display certain signals 104 in conjunction with one another, either in a real-time or recorded manner, in order for a direct comparison of the selected signals over the specifies time period. This, in turn, allows for the user to readily determine any observable correlations in the cardiac and neurological signals 104 being recorded in order to diagnose and treat any observed cardioneurogenic disease or condition.

The written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A physiological data acquisition system for obtaining information from sensors operably connected to the system, the system comprising:
a) an amplifier;
b) a first set of leads operably connected between a first set of sensors and the amplifier for transmitting a first set of physiological signals from the first set of sensors to the amplifier;
c) a second set of leads operably connected between a second set of sensors and the amplifier for transmitting a second set of physiological signals from the second set of sensors to the amplifier; and
d) a CPU operably connected to the amplifier to monitor the first set of signals and the second set of signals output from the amplifier to the CPU.

2. The system of claim 1 wherein the amplifier synchronizes the first set of physiological signals and the second set of physiological signals.

3. The system of claim 2 wherein the first set of sensors are EEG sensors.

4. The system of claim 2 wherein the first set of sensors are ECG sensors.

5. The system of claim 2 wherein the first set of sensors are intracardiac sensors.

6. The system of claim 2 wherein the device comprises only a single amplifier.

7. The system of claim 2 wherein the amplifier synchronizes the first set of signals and the second set of signals with respect to time.

8. The system of claim 1 wherein the CPU is configured to activate a recording of one of the first or second set of signals in response to the detection of an event detected by the CPU in the other of the first or second set of signals.

9. The system of claim 8 wherein the CPU is configured to activate a recording of a set of neurological signals in response to the detection of an event in a set of cardiac signals.

10. The system of claim 8 wherein the CPU is configured to activate a recording of a set of cardiac signals in response to the detection of an event in a set of neurological signals.

11. The system of claim 1 further comprising a display operably connected to the CPU and on which the first set of signals and the second set of signals can be illustrated.

12. The system of claim 11 wherein the CPU is configured to analyze the first set of signals or the second set of signals and provide a patient status value representative of the current status of a patient being monitored by the system that is illustrated on the display.

13. A method for diagnosing and treating a cardioneurogenic condition, the method comprising the steps of:
a) providing a physiological data acquisition system including an amplifier, a first set of leads operably connected between a first set of sensors and the amplifier for transmitting a first set of physiological signals from the first set of sensors to the amplifier, a second set of leads operably connected between a second set of sensors and the amplifier for transmitting a second set of physiological signals from the second set of sensors to the amplifier and a CPU operably connected to the amplifier to monitor the first set of signals and the second set of signals output from the amplifier;
b) synchronizing the first set of signals and the second set of signals in the amplifier with respect each other; and
c) recording at least one of the synchronized first set of signals and second set of signals for review and diagnosis of the cardioneurogenic condition.

14. The method of claim 13 wherein the step of synchronizing the first set of signals and the second set of signals comprises time synchronizing the first set of a signals and the second set of signals with respect to one another.

15. The method of claim 13 wherein the step of recording at least one of the first set of signals or the second set of signals comprises the steps of
a) recording one of the first set or second set of signals;
b) monitoring the recorded first set or second set of signals to determine if a triggering event has occurred; and
c) optionally recording the other of the first set or second set of signals if a triggering event has occurred.

16. The method of claim 15 wherein the triggering event is selected from the group consisting of a cardiac event and a neurological event.

17. The method of claim 13 wherein the step of recording at least one of the synchronized first set of signals and second set of signals comprises dynamically assigning an input of the amplifier to receive the at least one of the synchronized first set of signals and second set of signals for recording.

18. The method of claim 13 further comprising the step of displaying the first set of signals and the second set of signals in a time synchronous manner.

19. A physiological data acquisition system for obtaining information on multiple physiological parameters from sensors operably connected to the system, the system comprising:
a) an amplifier configured to receive and output signals representative of the multiple physiological parameters in a temporally synchronized format; and
b) a CPU operably connected to the amplifier to monitor the signals from the amplifier.

20. The system of claim 19 wherein the CPU is configured to activate a recording of signals received for one physiological parameter based on a triggering event determined from signals received for another physiological parameter.
